(19) United States
(12) Patent Application Publication
(10) Pub. No.: US 2006/0188984 A1
(43) Pub. Date: Aug. 24, 2006

(54) Method of providing readily available cellular material derived from cord blood, and a composition thereof

(76) Inventor: Donnie Rudd, Sugar Land, TX (US)

Correspondence Address: LADAS & PARRY LLP, 224 SOUTH MICHIGAN AVENUE, SUITE 1600, CHICAGO, IL, 60604, US

(21) Appl. No.: 11/340,114

(22) Filed: Jan. 26, 2006

Related U.S. Application Data
(60) Provisional application No. 60/647,588, filed on Jan. 27, 2005.

(51) Int. Cl. C12N 5/00; C12N 5/08; C12N 5/02

(52) U.S. Cl. 435/366; 435/372; 435/374

(57) ABSTRACT

The present invention is directed to the TVEMF-expansion of mammalian cord blood stem cells, preferably CD34+/CD38− cells, to compositions resulting from the TVEMF-expanded cells, and to a method of treating disease or repairing tissue with the compositions. Various benefits and advantages to the compositions of the present invention are discussed herein.

### CROSS-REFERENCE TO RELATED APPLICATIONS

The present application claims priority from U.S. Ser. No. 60/647,588, filed Jan. 27, 2005, and entitled “Method of Providing Readily Available Cellular Material Derived from Cord Blood”.

### FIELD OF THE INVENTION

The present invention is directed to cord blood stem cells prepared in a TVEMF-bioreactor, and to the process for such preparation, compositions thereof, and methods of treating a mammal with the cells or compositions.

### BACKGROUND OF THE INVENTION

Regeneration of human tissue has long been a desire of the medical community. Thus far, repair of human tissue has been accomplished largely by transplantations of like tissue from a donor. Beginning essentially with the kidney transplant from one of the Herrick twins to the other and later made world famous by South African Doctor Christian Barnard's transplant of a heart from Denise Darval to Louis Washkansky on Dec. 3, 1967, tissue transplantation became a widely accepted method of extending life in terminal patients.

Transplantation of human tissue, from its first use, encountered major problems, primarily tissue rejection due to the body's natural immune system. This often caused the use of tissue transplantation to have a limited prolongation of life (Washkansky lived only 18 days past the surgery).

In order to overcome the problem of the body's immune system, numerous anti-rejection drugs (e.g. Imuran, Cyclosporine) were soon developed to suppress the immune system and thus prolong the use of the tissue prior to rejection. However, the rejection problem has continued creating the need for an alternative to tissue transplantation.

Bone marrow transplantation was also used, and is still the procedure of choice for treatment of some illnesses, such as leukemia, to repair certain tissues such as bone marrow, but bone marrow transplantation also has problems. It requires a match from a donor (found less than 50% of the time); it is painful, expensive, and risky. Consequently, an alternative to bone marrow transplantation is highly desirable. Transplantation of tissue stem cells such as the transplantation of liver stem cells found in U.S. Pat. No. 6,129,911 have similar limitations rendering their widespread use questionable.

In recent years, researchers have experimented with the use of pluripotent embryonic stem cells as an alternative to tissue transplant. The theory behind the use of embryonic stem cells has been that they can theoretically be utilized to regenerate virtually any tissue in the body. The use of embryonic stem cells for tissue regeneration, however, has also encountered problems. Among the more serious of these problems are that transplanted embryonic stem cells have limited controllability, they sometimes grow into tumors, and the human embryonic stem cells that are available for research would be rejected by a patient's immune system (Nature, Jun. 17, 2002: Pearson, “Stem Cell Hopes Double”, news@nature.com, published online: 21 Jun. 2002). Further, widespread use of embryonic stem cells is so burdened with ethical, moral, and political concerns that its widespread use remains questionable.

Cord blood has been the focus of several areas of research. The pluripotent nature of stem cells was first discovered from an adult stem cell found in bone marrow. Verfaille, C. M. et al., Pluripotency of mesenchymal stem cells derived from adult marrow. Nature 417, published online 20 June; doi: 10.1038/nature00900, (2002) cited by Pearson, H. Stem cell hopes double. news@nature.com, published online: 21 Jun. 2002; doi: 10.1038/news020617-11. Pluripotent CD34+ stem cells and oligopotent lymphoid progenitor cells have been found in umbilical cord blood and have been shown to differentiate into cell types such as lymphoid natural killer cells. Perez S. A. et al., A novel myeloid-like NK cell progenitor in human umbilical cord blood. Blood 101(9):3444-50 (May 1, 2003). In addition, B cell progenitors have been discovered to reside not only in bone marrow, but also in cord blood. Sanz E. et al., Human cord blood CD34+Pax-5+ B-cell progenitors: single-cell analyses of their gene expression profiles. Blood 101(9):4324-30 (May 1, 2003).

Boyse et al., U.S. Pat. No. 6,569,427 B1, discloses the cryopreservation and usefulness of cryopreserved fetal or neonatal blood in the treatment or prevention of various diseases and disorders such as anemias, malignancies, autoimmune disorders, and various immune dysfunctions an deficiencies. Boyse also discloses the use of hematopoietic reconstitution in gene therapy with the use of a heterologous gene sequence. The Boyse disclosure stops short, however, of expansion of cells for therapeutic uses. C or Cell, a cord blood bank, provides statistics on expansion, cryopreservation, and transplantation of umbilical cord blood stem cells. “Expansion of Umbilical Cord Blood Stem Cells”, Information Sheet Umbilical Cord Blood, C or Cell, Inc. (2003). One expansion process discloses utilizing a bioreactor with a central collagen based matrix. Research Center Julich: Blood Stem Cells from the Bioreactor. Press release May 17, 2001. In addition, freezing cord blood cells before or after expansion has been shown to have no effect on the expansion capabilities of stem cells. Lazzari, L. et al., Evaluation of the effect of cryopreservation on ex vivo expansion of hematopoitic progenitors from cord blood. Bone Marrow Trans. 28:693-698(2001).

Cord blood has been found to provide better reconstitution of the hematopoietic reservoir as compared to bone marrow. Frassoni F. et al., Cord blood transplantation provides better reconstitution of hematopoeitic reservoir as compared to bone marrow transplantation. Blood (Apr. 3, 2003). See also First Unrelated Stem Cell Transplant Performed in Atlanta Dec. 12, 1998-1 year update. Bone Marrow and Cord Blood Stem Cell Transplant, The Sickle Cell Information Center (1999).

Research continues in an effort to elucidate the molecular mechanisms involved in the expansion of stem cells. For example, the C or Cell article discloses that a signal molecule named Delta-1 aids in the development of cord blood stem cells. Ohishi K. et al.: Delta-1 enhances marrow and thymus repopulating ability of human CD34+/CD38− cord blood cells. Clin. Invest. 110:1165-1174 (2002).

Throughout this application, the term “cord blood cells” means blood cells derived from the umbilical cord and/or the placenta of a fetus or infant.

Although cord blood cells are defined as adult, or somatic, stem cells, several factors make cord blood cells, and in particular cord blood stem cells, unique.

First, cord blood is primitive. Cord blood stem cells are young; they may have more plasticity than older cells, meaning they can give rise to a greater variety of specialized cells. They are also more likely to be healthier cells because they have had fewer opportunities to be affected by damaging environmental toxins that may change DNA. Furthermore, because they are young, cord blood stem cells can better integrate into the recipient patient and are less likely to cause graft vs. host disease (GvHD) or cell rejection. Also because they are young, cord blood stem cells may be considered a little less stable than adult-aged peripheral blood stem cells, for instance because the cord blood stem cells are still relatively new and have been in a very protected environment. Cord blood stem cells may therefore be more susceptible to damage, for instance from cryopreservation, than more aged stem cells.

Second, cord blood is stem cell-rich. Cord blood contains white blood cells (including mononuclear cells; for the purposes of this invention, a mononuclear cell is a cells having only one nucleus) and red blood cells. Typically, approximately 1-2% of cord blood mononuclear cells are stem cells. This makes cord blood one of the richest sources of stem cells. Cord blood collected from a Caesarean section is typically even a little richer in stem cells than cord blood collected immediately after vaginal birth. It is also easier to isolate stem cells in cord blood as opposed to other tissues. While adult stem cells can be found in numerous mature tissues, they are found in lesser quantities and are harder to locate.

Third, and finally, cord blood is an available source of stem cells. Adult stem cell transplants from body tissue such as bone marrow are not readily available. Cord blood banking provides a source of readily available stem cells. A cord blood collection from a typical human infant immediately after birth will typically yield 50 to 100 ml cord blood.

The umbilical cord, which contains cord blood, is the cord that connects a fetus to a maternal placenta, providing nutrients and removing wastes. The umbilical cord is a cordlike structure about 22 in. (56 cm) long, extending from the abdominal wall of the fetus to the maternal placenta.

The main function of the umbilical cord is to carry nourishment and oxygen from the placenta to the fetus and return waste products to the placenta from the fetus. Essentially, the umbilical cord is a cord like structure formed by, and integral with, the fetus' membrane at one end, with the other end terminating in the placenta. Enclosed within the cord is a mucoid jelly which houses one vein which carries oxygenated blood to the fetus and two arteries which carry un-oxygenated blood away from the fetus.

Blood is carried from the fetus along the umbilical cord and into the placenta. In the placenta in vivo, cord blood is brought into close proximity with the mother's blood such that oxygen, nutrients, and antibodies diffuse from the mother's blood into the cord blood. Waste materials from the fetus pass into the mother's blood, via the two un-oxygenated arteries. The cord blood, which has been enriched with nutrients, oxygenated, and cleaned of waste, is then carried back to the fetus by the vein that carries oxygenated blood through the umbilical cord.

After birth, the umbilical cord is clamped off and cut. The stump that is attached to the infant after the cord is cut off, eventually withers and drops off, leaving the scar known as the navel.

Because cord blood is especially rich in stem cells some parents choose to save it in special cord blood banks. The cord blood stem cells contained therein can be used in case of future need as a transplant alternative to bone marrow. Studies have shown that even people not related to the cord blood donor (genetically mismatched) may benefit from transplants of cord blood in combating leukemia and other cancers without eliciting an immune reaction rejecting the cord blood cells.

There are two typical ways of collecting cord blood; blood bag collection and syringe collection. Blood bag collection involves a health care provider inserting a needle into the umbilical vein and, with the assistance of gravity, draining the blood into a bag. Once the blood has stopped flowing, the bag will be sealed and labeled by the health care provider. This method is usually done before the placenta is delivered.

Syringe collection is similar to blood bag collection except that the cord blood is drawn into syringes containing anticoagulants (a substance that prevents the blood from clotting). The blood is stored in the syringes instead of in blood bags. This method can be done before or after the placenta is delivered. It is thought to be a more reliable way of collecting blood than blood bag collection. It also allows for more blood to be collected than is possible with blood bag collection. Regardless of which method is utilized, or whether another process for collecting cord blood is utilized, the whole process of collection may take as little as five minutes to perform, or even less. Preferably, the cord blood is collected within 10 to 15 minutes after birth. Waiting longer than this may result in less cord blood being collected, and therefore, fewer cord blood stem cells collected.

In the case of cord blood banking, or storage, once the cord blood arrives at the storage facility the cord blood is tested to make sure it does not carry any infectious or genetic diseases, like hepatitis, HIV/AIDS, leukemia, or an immune disorder. If there are any such problems with the cord blood, it may either be considered unsuitable for storage, or, in some instances, the blood may still be stored with the associated risks noted. If the blood is needed in the future parents can assess whether or not the need for the cord blood stem cells outweighs the associated risks carried with the cord blood.

Cord blood that will be stored typically goes through a series of processing before being banked. First, the cord blood is separated into its parts; white blood cells, red blood cells, and plasma. This is either done in a centrifuge (an apparatus that spins the container of blood until the blood is divided) or by sedimentation (the process of injecting sediment into the container of blood causing the blood to separate). Second, once the cord blood is divided with the red blood cells (RBC) on the bottom, white blood cells (WBC) in the middle, and the plasma on top, the white blood cells are removed for storage. The middle layer, also known as the “buffy coat” contains the cord blood stem cells of interest; the other parts of the blood are not needed. For some banks, this will be the extent of their processing. However, other banks will go on to process the buffy coat by removing the mononuclear cells (in this case, a subset of white blood cells) from the WBC. While not everyone agrees with this method, there is less to store and less cryogenic nitrogen is needed to store the cells.

It is preferable to remove the RBC from the cord blood sample. While people may have the same HLA type (which is needed for the transplanting of stem cells), they may not have the same blood type. By removing the RBC, adverse reactions to a stem cell transplant can be minimized. By eliminating the RBC, therefore, the stem cell sample has a better chance of being compatible with more people. RBC can also burst when they are thawed, releasing free hemoglobin. This type of hemoglobin can seriously affect the kidneys of people receiving a transplant. Additionally, the viability of the stem cells are reduced when RBC rupture.

Prior to the cells being frozen, they may be expanded (that is, increased in number, not size).

Once the RBC's are removed the cells will begin to be preserved and frozen for long-term storage. However, this must be done slowly and carefully in order not to damage the stem cells. Before the blood cells are frozen, they are first mixed in a solution to help prevent them from being damaged while frozen. This solution is referred to as the cryopreservative, cryopreservation solvent or cryoprotectant. Once the expanded cells are in cryopreservative, they are slowly frozen so as to guard the cells against damage.

Once frozen (generally to a temperature of about −196 C), the cells are transferred to a permanent storage freezer. While in this freezer, they will remain frozen in either liquid or vapor nitrogen. Different types of freezers are commonly used to preserve cord blood stem cells. One type is the “BioArchive” freezer. This machine not only freezes the blood, but also inventories it and manages up to 3,626 blood bags. It has a robotic arm that will retrieve the specified blood sample when required. This ensures that no other samples are disturbed or exposed to warmer temperatures. Other types presently commercially available include, but are not limited to, Sanyo Model MDF-1155ATN-152C and Model MDF-2136 ATN-135C, and Princeton CryoTech TEC 2000.

Expansion of the cord blood stem cells may take several days. In a situation where it is important to have an immediate supply of cord blood stem cells, such as a life-or-death situation or in the case of a traumatic injury, especially if research needs to be accomplished prior to reintroduction of the cells, several days may not be available to await for the expansion of the cord blood stem cells. It is particularly desirable, therefore, to have such expanded cord blood stem cells available from birth forward in anticipation of an emergency where every minute in delaying treatment can mean the difference in life or death.

There is a need, therefore, to provide a method and process of repairing human tissue that is not based on organ transplantation, bone marrow transplantation, or embryonic stem cells, and yet provides a composition of expanded cord blood stem cells, unlikely to elicit an immune response, for use in a matter of hours rather than days.

### SUMMARY OF THE INVENTION

The present invention relates in part to cord blood stem cells from a mammal, preferably human, wherein said cord blood stem cells are TVEMF-expanded. The present invention also relates to cord blood stem cells from a mammal, preferably human, wherein said cord blood stem cells are in a number per volume that is at least 7 times greater than naturally-occurring cord blood; and wherein the cord blood stem cells have a three-dimensional geometry and cell-to-cell support and cell-to-cell geometry that is essentially the same as stem cells of naturally-occurring cord blood. Such cells are preferably made by the TVEMF-expansion process described herein. The invention also relates to compositions comprising these cells, with other components added as desired, including pharmaceutically acceptable carriers, cryopreservatives, and cell culture media.

The present invention also relates to a process for preparing cord blood stem cells and cord blood stem cell compositions by placing a cord blood mixture in a culture chamber of a TVEMF-bioreactor; and subjecting the cord blood mixture to a TVEMF and TVEMF-expanding the cord blood stem cells to prepare the cord blood stem cell composition. Preferably, the TVEMF applied to the cells is from about 0.05 to about 6.0 gauss.

The present invention also relates to a method of treating a mammal with the cord blood stem cells and cord blood stem cell compositions of the present invention. Such treatment may be for tissue repair and regeneration, to treat a disease, or any other uses discussed throughout this application. Also comprised herein is a composition and method for the treatment of any of the diseases defined herein, or for the repair of tissue or organ, comprising the cord blood stem cell compositions of the present invention. Also comprised herein is use of a composition of the present invention for the preparation of a medicament for the treatment of any of the diseases discussed herein, or for the repair or regeneration of tissue as described herein.

### BRIEF DESCRIPTION OF THE DRAWINGS

In the drawings,

FIG. 1 schematically illustrates a preferred embodiment of a culture carrier flow loop of a bioreactor;

FIG. 2 is an elevated side view of a preferred embodiment of a TVEMF-bioreactor of the invention;

FIG. 3 is a side perspective of a preferred embodiment of the TVEMF-bioreactor of FIG. 2;

FIG. 4 is a vertical cross sectional view of a preferred embodiment of a TVEMF-bioreactor;

FIG. 5 is a vertical cross sectional view of a TVEMF-bioreactor;

FIG. 6 is an elevated side view of a time varying electromagnetic force device that can house, and provide a time varying electromagnetic force to, a bioreactor;

FIG. 7 is a front view of the device shown in FIG. 6; and

FIG. 8 is a front view of the device shown in FIG. 6, further showing a bioreactor therein.

### DETAILED DESCRIPTION OF THE DRAWINGS

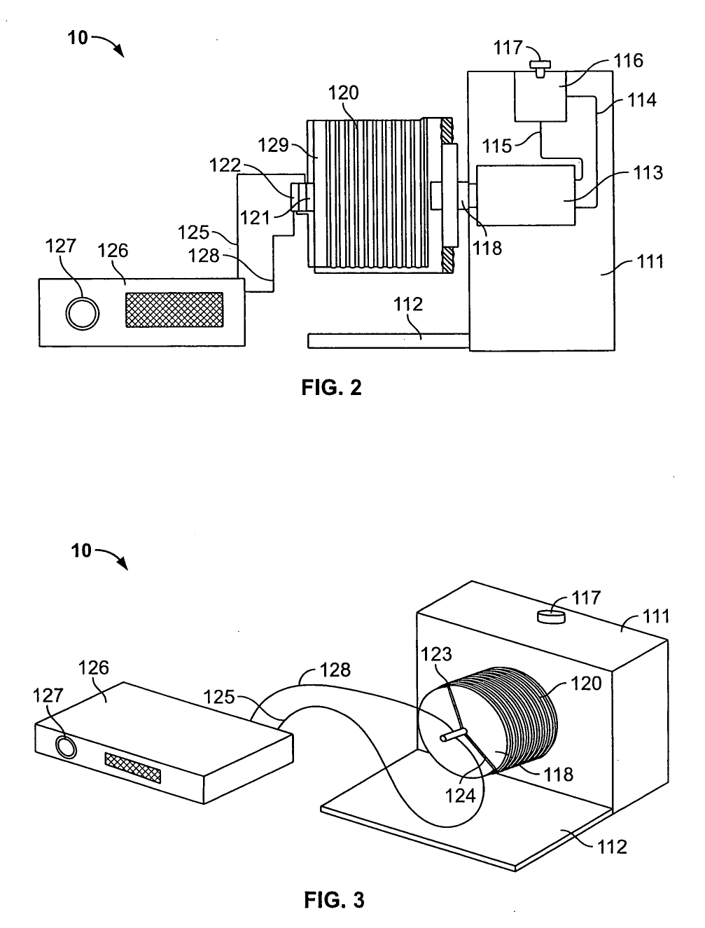

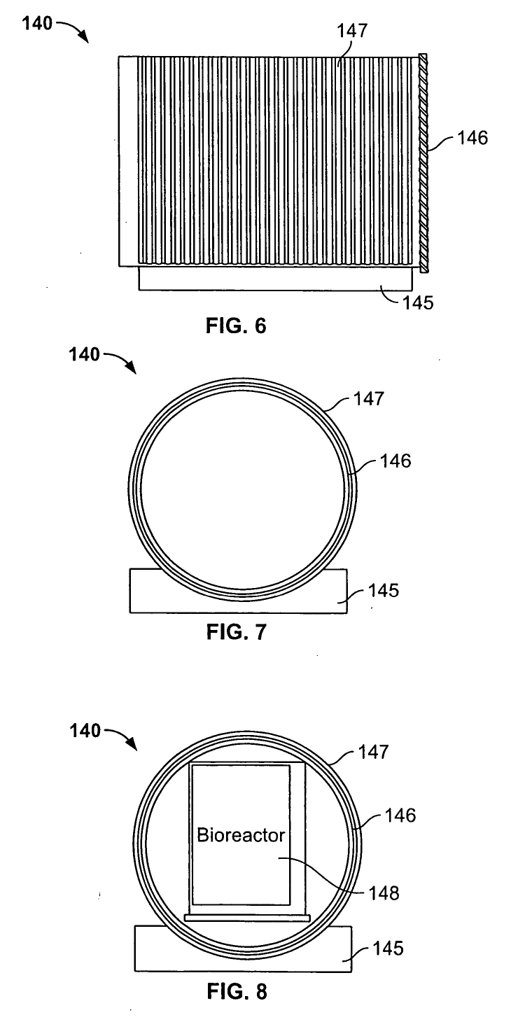

In the simplest terms, a rotating TVEMF-bioreactor comprises a cell culture chamber and a time varying electromagnetic force source. In operation, a cord blood mixture is place into the cell culture chamber. The cell culture chamber is rotated over a period of time during which a time varying electromagnetic force is generated in the chamber by the time varying electromagnetic force source. Upon completion of the time, the expanded cord blood mixture is removed from the chamber. In a more complex TVEMF-bioreactor system, the time varying electromagnetic force source can be integral to the TVEMF-bioreactor, as illustrated in FIGS. 2-5, but can also be adjacent to a bioreactor as in FIGS. 6-8. Furthermore, a fluid carrier, which provides sustenance to the cells, can be periodically refreshed and removed. Preferred TVEMF-bioreactors are described herein.

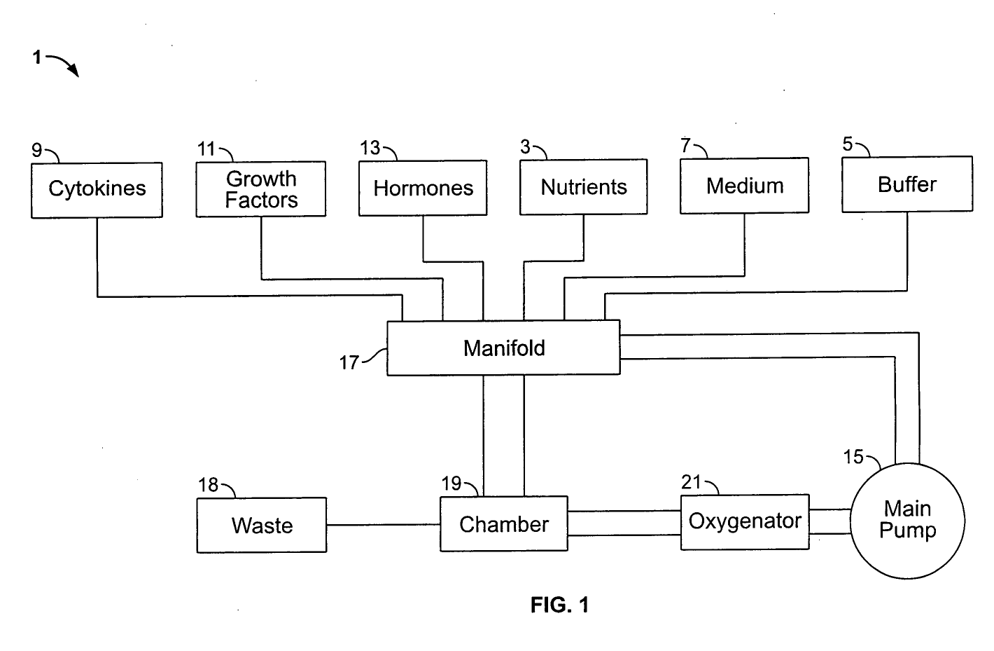

Referring now to FIG. 1, illustrated is a preferred embodiment of a culture carrier flow loop 1 in an overall bioreactor culture system for growing mammalian cells having a cell culture chamber 19, preferably a rotating cell culture chamber, an oxygenator 21, an apparatus for facilitating the directional flow of the culture carrier, preferably by the use of a main pump 15, and a supply manifold 17 for the selective input of such culture carrier requirements as, but not limited to, nutrients 3, buffers 5, fresh medium 7, cytokines 9, growth factors 11, and hormones 13. In this preferred embodiment, the main pump 15 provides fresh fluid carrier to the oxygenator 21 where the fluid carrier is oxygenated and passed through the cell culture chamber 19. The waste in the spent fluid carrier from the cell culture chamber 19 is removed and delivered to the waste 18 and the remaining cell culture carrier is returned to the manifold 17 where it receives a fresh charge, as necessary, before recycling by the pump 15 through the oxygenator 21 to the cell culture chamber 19.

In the culture carrier flow loop 1, the culture carrier is circulated through the living cell culture in the chamber 19 and around the culture carrier flow loop 1, as shown in FIG. 1. In this loop 1, adjustments are made in response to chemical sensors (not shown) that maintain constant conditions within the cell culture reactor chamber 19. Controlling carbon dioxide pressures and introducing acids or bases corrects pH. Oxygen, nitrogen, and carbon dioxide are dissolved in a gas exchange system (not shown) in order to support cell respiration. The closed loop 1 adds oxygen and removes carbon dioxide from a circulating gas capacitance. Although FIG. 1 is one preferred embodiment of a culture carrier flow loop that may be used in the present invention, the invention is not intended to be so limited. The input of culture carrier requirements such as, but not limited to, oxygen, nutrients, buffers, fresh medium, cytokines, growth factors, and hormones into a bioreactor can also be performed manually, automatically, or by other control means, as can be the control and removal of waste and carbon dioxide.

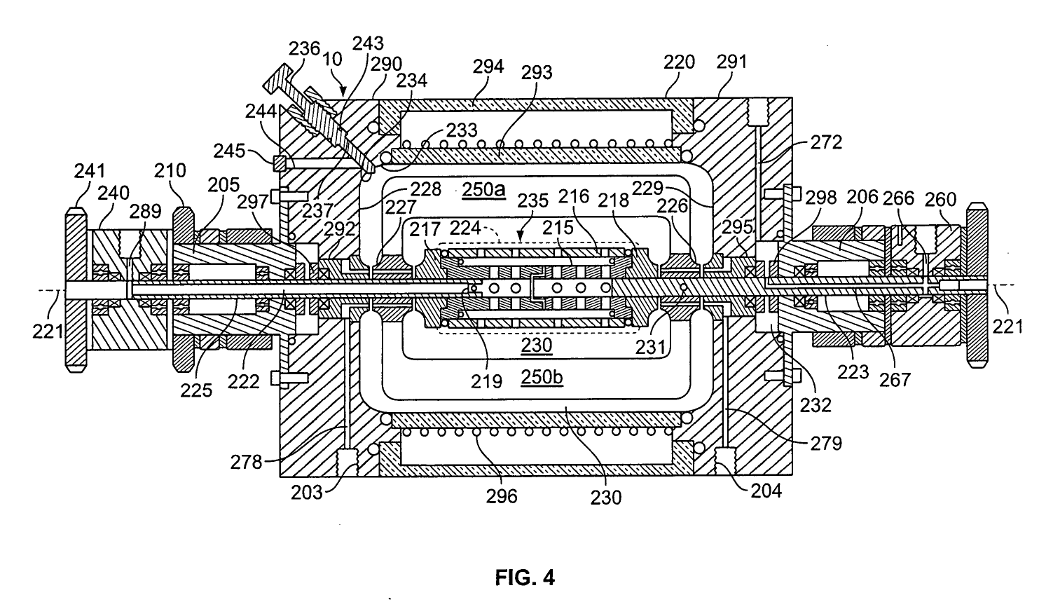

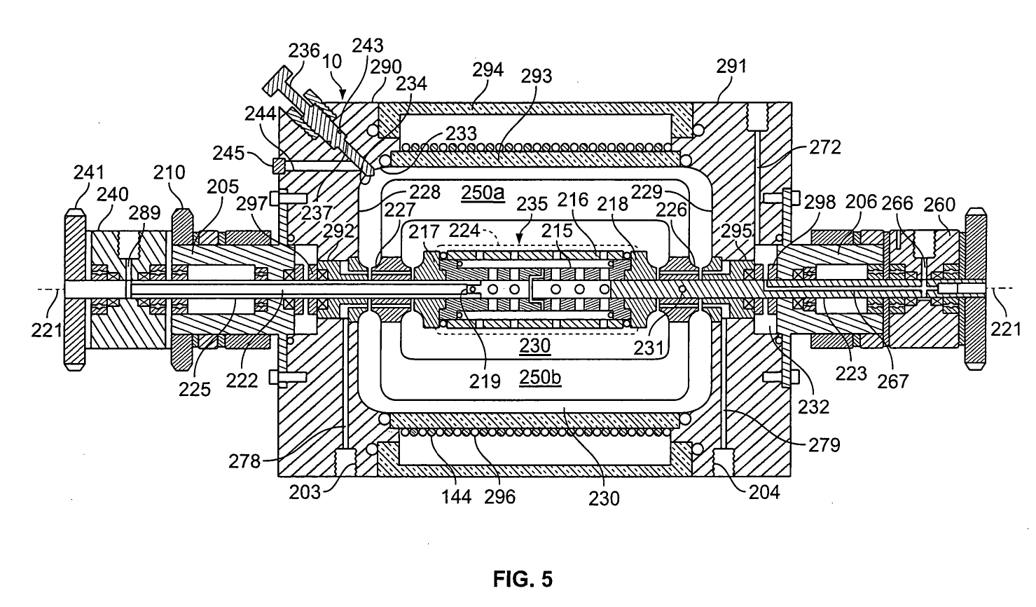

FIGS. 2 and 3 illustrate a preferred embodiment of a TVEMF-bioreactor 10 with an integral time varying electromagnetic force source. FIG. 4 is a cross section of a rotatable TVEMF-bioreactor 10 for use in the present invention in a preferred form. The TVEMF-bioreactor 10 of FIG. 4 is illustrated with an integral time varying electromagnetic force source. FIG. 5 also illustrates a preferred embodiment of a TVEMF-bioreactor with an integral time varying electromagnetic force source. FIGS. 6-8 show a rotating bioreactor with an adjacent time varying electromagnetic force source.

Turning now to FIG. 2, illustrated in FIG. 2 is an elevated side view of a preferred embodiment of a TVEMF-bioreactor 10 of the present invention. FIG. 2 comprises a motor housing 111 supported by a base 112. A motor 113 is attached inside the motor housing 111 and connected by a first wire 114 and a second wire 115 to a control box 116 that has a control means therein whereby the speed of the motor 113 can be incrementally controlled by turning the control knob 117. The motor housing 111 has a motor 113 inside set so that a motor shaft 118 extends through the housing 111 with the motor shaft 118 being longitudinal so that the center of the shaft 118 is parallel to the plane of the earth at the location of a longitudinal chamber 119, preferably made of a transparent material including, but not limited to, plastic.

In this preferred embodiment, the longitudinal chamber 119 is connected to the shaft 118 so that the chamber 119 rotates about its longitudinal axis with the longitudinal axis parallel to the plane of the earth. The chamber 119 is wound with a wire coil 120. The size of the wire coil 120 and number of times it is wound are such that when a square wave current preferably of from 0.1 mA to 1000 mA is supplied to the wire coil 120, a time varying electromagnetic force preferably of from 0.05 gauss to 6 gauss is generated within the chamber 119. The wire coil 120 is connected to a first ring 121 and a second ring 122 at the end of the shaft 118 by wires 123 and 124. These rings 121, 122 are then contacted by a first electromagnetic delivery wire 125 and a second electromagnetic delivery wire 128 in such a manner that the chamber 119 can rotate while the current is constantly supplied to the coil 120. An electromagnetic generating device 126 is connected to the wires 125, 128. The electromagnetic generating device 126 supplies a square wave to the wires 125, 128 and coil 120 by adjusting its output by turning an electromagnetic generating device knob 127.

FIG. 3 is a side perspective view of the TVEMF-bioreactor 10 shown in FIG. 2 that may be used in the present invention.

Turning now to the rotating TVEMF-bioreactor 10 illustrated in FIG. 4 with a culture chamber 230 which is preferably transparent and adapted to contain a cord blood mixture therein, further comprising an outer housing 220 which includes a first 290 and second 291 cylindrically shaped transverse end cap member having facing first 228 and second 229 end surfaces arranged to receive an inner cylindrical tubular glass member 293 and an outer tubular glass member 294. Suitable pressure seals are provided. Between the inner 293 and outer 294 tubular members is an annular wire heater 296 which is utilized for obtaining the proper incubation temperatures for cell growth. The wire heater 296 can also be used as a time varying electromagnetic force device to supply a time varying electric field to the culture chamber 230 or, as depicted in FIG. 5, a separate wire coil 144 can be used to supply a time varying electromagnetic force. The first end cap member 290 and second end cap member 291 have inner curved surfaces adjoining the end surfaces 228, 229 for promoting smoother flow of the mixture within the chamber 230. The first end cap member 290, and second end cap member 291 have a first central fluid transfer journal member 292 and second central fluid transfer journal member 295, respectively, that are rotatably received respectively on an input shaft 223 and an output shaft 225. Each transfer journal member 294, 295 has a flange to seat in a recessed counter bore in an end cap member 290, 291 and is attached by a first lock washer and ring 297, and second lock washer and ring 298 against longitudinal motion relative to a shaft 223, 225. Each journal member 294, 295 has an intermediate annular recess that is connected to longitudinally extending, circumferentially arranged passages. Each annular recess in a journal member 292, 295 is coupled by a first radially disposed passage 278 and second radially disposed passage 279 in an end cap member 290 and 291, respectively, to first input coupling 203 and second input coupling 204. Carrier in a radial passage 278 or 279 flows through a first annular recess and the longitudinal passages in a journal member 294 or 295 to permit access carrier through a journal member 292, 295 to each end of the journal 292, 295 where the access is circumferential about a shaft 223, 225.

Attached to the end cap members 290 and 291 are a first tubular bearing housing 205, and second tubular bearing housing 206 containing ball bearings which relatively support the outer housing 220 on the input 223 and output 225 shafts. The first bearing housing 205 has an attached first sprocket gear 210 for providing a rotative drive for the outer housing 220 in a rotative direction about the input 223 and output 225 shafts and the longitudinal axis 221. The first bearing housing 205, and second bearing housing 206 also have provisions for electrical take out of the wire heater 296 and any other sensor.

The inner filter assembly 235 includes inner 215 and outer 216 tubular members having perforations or apertures along their lengths and have a first 217 and second 218 inner filter assembly end cap member with perforations. The inner tubular member 215 is constructed in two pieces with an interlocking centrally located coupling section and each piece attached to an end cap 217 or 218. The outer tubular member 216 is mounted between the first 217 and second inner filter assembly end caps.

The end cap members 217, 218 are respectively rotatably supported on the input shaft 223 and the output shaft 225. The inner member 215 is rotatively attached to the output shaft 225 by a pin and an interfitting groove 219. A polyester cloth 224 with a ten-micron weave is disposed over the outer surface of the outer member 216 and attached to O-rings at either end. Because the inner member 215 is attached by a coupling pin to a slot in the output drive shaft 225, the output drive shaft 225 can rotate the inner member 215. The inner member 215 is coupled by the first 217 and second 218 end caps that support the outer member 216. The output shaft 225 is extended through bearings in a first stationary housing 240 and is coupled to a first sprocket gear 241. As illustrated, the output shaft 225 has a tubular bore 222 that extends from a first port or passageway 289 in the first stationary housing 240 located between seals to the inner member 215 so that a flow of fluid carrier can be exited from the inner member 215 through the stationary housing 240.

Between the first 217 and second 218 end caps for the inner member 235 and the journals 292, 295 in the outer housing 220, are a first 227 and second 226 hub for the blade members 50a and 50b. The second hub 226 on the input shaft 223 is coupled to the input shaft 223 by a pin 231 so that the second hub 226 rotates with the input shaft 223. Each hub 227, 226 has axially extending passageways for the transmittal of carrier through a hub.

The input shaft 223 extends through bearings in the second stationary housing 260 for rotatable support of the input shaft 223. A second longitudinal passageway 267 extends through the input shaft 223 to a location intermediate of retaining washers and rings that are disposed in a second annular recess 232 between the faceplate and the housing 260. A third radial passageway 272 in the second end cap member 291 permits fluid carrier in the recess to exit from the second end cap member 291. While not shown, the third passageway 272 connects through piping and a Y joint to each of the passages 278 and 279.

A sample port is shown in FIG. 4, where a first bore 237 extending along a first axis intersects a corner 233 of the chamber 230 and forms a restricted opening 234. The bore 237 has a counter bore and a threaded ring at one end to threadedly receive a cylindrical valve member 236. The valve member 236 has a complimentarily formed tip to engage the opening 234 and protrude slightly into the interior of the chamber 230. An O-ring 243 on the valve member 236 provides a seal. A second bore 244 along a second axis intersects the first bore 237 at a location between the O-ring 243 and the opening 234. An elastomer or plastic stopper 245 closes the second bore 244 and can be entered with a hypodermic syringe for removing a sample. To remove a sample, the valve member 236 is backed off to access the opening 234 and the bore 244. A syringe can then be used to extract a sample and the opening 234 can be reclosed. No outside contamination reaches the interior of the TVEMF-bioreactor 10.

In operation, carrier is input to the second port or passageway 266 to the shaft passageway and thence to the first radially disposed 278 and second radially disposed passageways 279 via the third radial passageway 272. When the carrier enters the chamber 230 via the longitudinal passages in the journals 292, 294 the carrier impinges on an end surface 228, 229 of the hubs 227, 226 and is dispersed radially as well as axially through the passageways in the hubs 227, 226. Carrier passing through the hubs 227, 226 impinges on the end cap members 217, 218 and is dispersed radially. The flow of entry fluid carrier is thus radially outward away from the longitudinal axis 221 and flows in a toroidal fashion from each end to exit through the polyester cloth 224 and openings in filter assembly 235 to exit via the passageways 266 and 289. By controlling the rotational speed and direction of rotation of the outer housing 220, chamber 230, and inner filter assembly 235 any desired type of carrier action can be obtained. Of major importance, however, is the fact that a clinostat operation can be obtained together with a continuous supply of fresh fluid carrier.

If a time varying electromagnetic force is not applied using the integral annular wire heater 296, it can be applied by another preferred time varying electromagnetic force source. For instance, FIGS. 6-8 illustrate a time varying electromagnetic force device 140 which provides an electromagnetic force to a cell culture in a bioreactor which does not have an integral time varying electromagnetic force, but rather has an adjacent time varying electromagnetic force device. Specifically, FIG. 6 is a preferred embodiment of a time varying electromagnetic force device 140. FIG. 6 is an elevated side perspective of the device 140 which comprises a support base 145, a cylinder coil support 146 supported on the base 145 with a wire coil 147 wrapped around the support 146. FIG. 7 is a front perspective of the time varying electromagnetic force device 140 illustrated in FIG. 6. FIG. 8 is a front perspective of the time varying electromagnetic force device 140, which illustrates that in operation, an entire bioreactor 148 is inserted into a cylinder coil support 146 which is supported by a support base 145 and which is wound by a wire coil 147. Since the time varying electromagnetic force device 140 is adjacent to the bioreactor 148, the time varying electromagnetic force device 140 can be reused. In addition, since the time varying electromagnetic force device 140 is adjacent to the bioreactor 148, the device 140 can be used to generate an electromagnetic force in all types of bioreactors, preferably rotating.

In operation, during TVEMF-expansion, a TVEMF-bioreactor 10 of the present invention contains a cord blood mixture in the cell culture chamber. During TVEMF-expansion, the speed of the rotation of the cord blood mixture-containing chamber may be assessed and adjusted so that the cord blood mixture remains substantially at or about the longitudinal axis. Increasing the rotational speed is warranted to prevent wall impact. For instance, an increase in the rotation is preferred if the cord blood stem cells in the cord blood mixture fall excessively inward and downward on the downward side of the rotation cycle and excessively outward and insufficiently upward on the upward side of the rotation cycle. Optimally, the user is advised to preferably select a rotational rate that fosters minimal wall collision frequency and intensity so as to maintain the cord blood stem cell three-dimensional geometry and their cell-to-cell support and cell-to-cell geometry. The preferred speed of the present invention is of from 5 to 120 RPM, and more preferably from 10 to 30 RPM.

The cord blood mixture may preferably be visually assessed through the preferably transparent culture chamber and manually adjusted. The assessment and adjustment of the cord blood mixture may also be automated by a sensor (for instance, a laser), which monitors the location of the cord blood stem cells within a TVEMF-bioreactor 10. A sensor reading indicating too much cell movement will automatically cause a mechanism to adjust the rotational speed accordingly.

Furthermore, in operation the present invention contemplates that an electromagnetic generating device is turned on and adjusted so that the square wave output generates the desired electromagnetic field in the cord blood mixture-containing chamber, preferably in a range of from 0.05 gauss to 6 gauss.

As various changes could be made in rotating bioreactors subjected to a time varying electromagnetic force as are contemplated in the present invention, without departing from the scope of the invention, it is intended that all matter contained in the above description be interpreted as illustrative and not limiting.

### DETAILED DESCRIPTION OF THE PREFERRED EMBODIMENTS OF THE INVENTION

The following definitions are meant to aid in the description and understanding of the defined terms in the context of the present invention. The definitions are not meant to limit these terms to less than is described throughout this application. Furthermore, several definitions are included relating to TVEMF—all of the definitions in this regard should be considered to complement each other, and not construed against each other.

As used throughout this application, the term “adult stem cell” refers to a pluripotent cell that is undifferentiated and that may give rise to more differentiated cells. With regard to the present invention, an adult stem cell is preferably CD34+/CD38−.

As used throughout this application, the term “cord blood” refers to blood from the umbilical cord and/or placenta of a fetus or infant. Cord blood is one of the richest sources of stem cells known. The term “cord” is not intended in any way to limit the term “cord blood” of this invention to blood from the umbilical cord; as explained throughout the application, the blood of a fetus' or infant's placenta is confluent with the blood of the umbilical cord. For the purposes of the present invention, there is no reason to distinguish between blood located at different parts of the same circulatory loop.

As used throughout this application, the term “cord blood cell” refers to a cell from cord blood. Cord blood cells capable of replication may undergo TVEMF-expansion in a TVEMF-bioreactor, and may be present in compositions of the present invention.

As used throughout this application, the term “cord blood stem cell” refers to an adult stem cell from cord blood. Cord blood stem cells are adult stem cells, also known as somatic stem cells, and are not embryonic stem cells derived directly from an embryo. Preferably, a cord blood stem cell of the present invention is a CD34+/CD38− cell.

As used throughout this application, the term “cord blood stem cell composition” refers to cord blood stem cells of the present invention, either in a number per volume at least 7 times greater than the naturally-occurring cord blood source and having the same or similar three-dimensional geometry and cell-to-cell geometry and cell-to-cell support as naturally-occuring cord blood stem cells, or having undergone TVEMF-expansion, maintaining the above mentioned geometry and support. With the cord blood stem cells is a carrier of some sort, whether a pharmaceutically acceptable carrier, plasma, blood, albumin, cell culture medium, growth factor, copper chelating agent, hormone, buffer, cryopreservative, or some other substance. Reference to naturally-occurring cord blood is preferably to compare cord blood stem cells of the present invention with their original cord blood source. However, if such a comparison is not available, then naturally-occurring cord blood may refer to average or typical characteristics of cord blood, preferably of the same mammalian species as the source of the cord blood stem cells of this invention.

As used throughout this application, the term “cord blood mixture” refers to a mixture of cord blood cells with a substance that allows the cells to expand, such as a medium for growth of cells, that will be placed in a TVEMF-bioreactor (for instance in a cell culture chamber). The cord blood cells may be present in the cord blood mixture simply by mixing whole cord blood with a substance such as a cell culture medium. Also, the cord blood mixture may be made with a cellular preparation from cord blood, as described throughout this application, containing cord blood stem cells. Preferably, the cord blood mixture comprises CD34+/CD38− cord blood stem cells and Dulbecco's medium (DMEM). Preferably, at least half of the cord blood mixture is a cell culture medium such as DMEM.

As used throughout this application, the term “TVEMF” refers to “Time Varying Electromagnetic Force”.

As used throughout this application, the term “TVEMF-bioreactor” refers to a rotating bioreactor to which TVEMF is applied, as described more fully in the Description of the Drawings, above. The TVEMF applied to a bioreactor is preferably in the range of 0.05 to 6.0 gauss, preferably 0.05-0.5 gauss. See for instance FIGS. 2, 3, 4 and 5 herein for examples (not meant to be limiting) of a TVEMF-bioreactor. In a simple embodiment, a TVEMF-bioreactor of the present invention provides for the rotation of an enclosed cord blood mixture at an appropriate gauss level (with TVEMF applied), and allows the cord blood cells (including stem cells) therein to expand. Preferably, a TVEMF-bioreactor allows for the exchange of growth medium (preferably with additives) and for oxygenation of the cord blood mixture. The TVEMF-bioreactor provides a mechanism for growing cells for several days or more. The TVEMF-bioreactor subjects cells in the bioreactor to TVEMF, so that TVEMF is passed through the cells, thus undergoing TVEMF-expansion.

As used throughout this application, the term “TVEMF-expanded cord blood cells” refers to cord blood cells increased in number per volume (ie concentration) after being placed in a TVEMF-bioreactor and subjected to a TVEMF of about 0.05 to 6.0. The increase in number of cells per volume is the result of cell replication in the TVEMF-bioreactor, so that the total number of cells in the bioreactor increase. The increase in number of cells per volume is expressly not due to a simple reduction in volume of fluid, for instance, reducing the volume of blood from 70 ml to 10 ml and thereby increasing the number of cells per ml.

As used throughout this application, the term “TVEMF-expanded cord blood stem cells” refers to cord blood stem cells increased in number per volume (ie concentration) after being placed in a TVEMF-bioreactor and subjected to a TVEMF of about 0.05 to 6.0 gauss. The increase in number of stem cells per volume is the result of cell replication in the TVEMF-bioreactor, so that the total number of stem cells in the bioreactor increase. The increase in number of stem cells per volume is expressly not due to a simple reduction in volume of fluid, for instance, reducing the volume of blood from 70 ml to 10 ml and thereby increasing the number of stem cells per ml.

As used throughout this application, the term “TVEMF-expanding” refers to the step of cells in a TVEMF-bioreactor replicating (splitting and growing) in the presence of TVEMF in a TVEMF-(rotating) bioreactor. Cord blood stem cells (preferably CD34+/CD38− stem cells) preferably replicate without undergoing further differentiation.

As used throughout this application, the term “TVEMF-expansion” refers to the process of increasing the number of cord blood cells in a TVEMF-bioreactor, preferably cord blood stem cells, by subjecting the cells to a TVEMF of about 0.05 to about 6.0 gauss. Preferably, the increase in number of cord blood cells, preferably cord blood stem cells, is at least 7 times the number per volume (ie concentration) of the original cord blood source. The expansion of cord blood stem cells in a TVEMF-bioreactor according to the present invention provides for cord blood stem cells that maintain, or have essentially the same, three-dimensional geometry and cell-to-cell support and cell-to-cell geometry as cord blood stem cells prior to TVEMF-expansion. Other aspects of TVEMF-expansion may also provide the exceptional characteristics of the cord blood stem cells of the present invention. Not to be bound by theory, TVEMF-expansion not only provides for high concentrations of cord blood stem cells that maintain their three-dimensional geometry and cell-to-cell support. Not to be bound by theory, TVEMF may affect some properties of stem cells during TVEMF-expansion, for instance up-regulation of genes promoting grown, or down regulation of genes preventing growty. Overall, TVEMF-expansion results in promoting growth but not differentiation overall.

As used throughout this application, the term “TVEMF-expanded cell” refers to a cell that has been subjected to the process of TVEMF-expansion.

As used throughout this application, the term “toxic substance” or related terms may refer to substances that are toxic to a cell, preferably a cord blood stem cell; or to a patient. In particular, the term toxic substance refers to dead cells, macrophages, as well as substances that may be unique or unusual in cord blood (for instance, sickle cells, maternal blood or maternal urine or other tissue or waste). Other toxic substances are discussed throughout this application. Removal of these substances from blood is well-known in the art.

Other statements referring to the above-defined terms or other terms used throughout this application are not meant to be limited by the above definitions, and may contribute to the definitions. Information relating to various aspects of this invention is provided throughout this application, and is not meant to be limited only to the section to which it is contained, but is meant to contribute to an understanding of the invention as a whole.

The present invention is related to providing a rapidly available source of TVEMF-expanded cord blood stem cells for repairing, replenishing and regenerating tissue in humans. This invention may be more fully described by the preferred embodiment(s) as hereinafter described, but is not intended to be limited thereto.

Operative Method—Preparing a TVEMF-Expanded Cord Blood Stem Cell Composition, and Using the Composition

In a preferred embodiment of this invention, a method is described for preparing TVEMF-expanded cord blood stem cells that can assist the body in repairing, replacing and regenerating tissue or be useful in research.

Cord blood is collected from a mammal, preferably a primate mammal, and more preferably a human, for instance as described throughout this application, and preferably according to the syringe method. Cord blood may also be collected in utero, for instance in life-threatening situations or extreme situations where a defect (for instance an ear defect) is apparent during the third trimester of pregnancy, so that cord blood stem cells may be expanded and readily available if needed at birth or soon after birth of the infant. Cord blood in utero would only be removed in an amount that would not be threatening to the unborn infant. The collection of cord blood according to this invention is not meant to be limiting, but can also include for instance other means of directly collecting mammalian cord blood, or indirectly collecting blood for instance by acquiring the blood from a commercial or other source, including for instance cryopreserved blood from a “blood bank”.

Preferably, red blood cells are removed from the cord blood and the remaining cells including cord blood stem cells are placed with an appropriate media in a TVEMF-bioreactor (see “cord blood mixture”) such as that described herein. In a more preferred embodiment of this invention, only the “buffy coat” (which includes cord blood stem cells, as discussed throughout this application) described above is placed in the TVEMF-bioreactor. Other embodiments include removing other non-stem cells and components of the cord blood, to prepare different cord blood preparation(s). Such a cord blood preparation may even have, as the only remaining cord blood component, CD34+/CD38− cord blood stem cells. Removal of non-stem cell types of cord blood cells may be achieved through negative separation techniques, such as but not limited to sedimentation and centrifugation. Many negative separation methods are well-known in the art. However, positive selection techniques may also be used, and are preferred in this invention. Methods for removing various components of the blood and positively selecting for CD34+/CD38− are known in the art, and may be used so long as they do not lyse or otherwise irreversibly harm the desired cord blood stem cells. For instance, an affinity method selective for CD34+/CD38− may be used. Preferably, a “buffy coat” as described above is prepared from cord blood, and the CD34+/CD38− cells therein separated from the buffy coat for TVEMF-expansion.

The collected cord blood, or desired cellular parts as discussed above, must be placed into a TVEMF-bioreactor for TVEMF-expansion to occur. As discussed above, the term “cord blood mixture” comprises a mixture of cord blood (or desired cellular part, for instance cord blood without red blood cells, or preferably CD34+/CD38− cord blood stem cells isolated from cord blood) with a substance that allows the cells to expand, such as a medium for growth of cells, that will be placed in a TVEMF-bioreactor. Cell culture media, media that allow cells to grow and expand, are well-known in the art. Preferably, the substance that allows the cells to expand is cell culture media, more preferably Dulbecco's medium. The components of the cell media must, of course, not kill or damage cells. Other components may also be added to the cord blood mixture prior to or during TVEMF-expansion. For instance, the cord blood may be placed in the bioreactor with Dulbecco's medium and further supplemented with 5% (or some other desired amount, for instance in the range of about 1% to about 10%) of human serum albumin. Other additives to the cord blood mixture, including but not limited to growth factor, copper chelating agent, cytokine, hormone and other substances that may enhance TVEMF-expansion may also be added to the cord blood outside or inside the bioreactor before being placed in the bioreactor. Preferably, the entire volume of a cord blood collection from one individual (preferably human cord blood in an amount of about 10 ml to about 100 ml, more preferably 50 ml to about 100 ml cord blood) is mixed with from about 25 ml to about 100 ml Dulbecco's medium (DMEM) and supplemented with 5% human serum albumin so that the total volume of the cord blood mixture is about 75 to about 200 ml when placed in the bioreactor. As a general rule, the more cord blood that may be collected, the better; if a collection from one individual results in more than 100 ml, the use of all of that valuable cord blood is preferred. Where a larger volume is available, for instance by pooling cord blood, more than one dose may be preferred. The use of a perfusion TVEMF-bioreactor is particularly useful when cord blood collections are pooled and TVEMF-expanded together.

The term “placed into a TVEMF-bioreactor” is not meant to be limiting—the cord blood mixture may be made entirely outside of the bioreactor and then the mixture placed inside the bioreactor. Also, the cord blood mixture may be entirely mixed inside the bioreactor. For instance, the cord blood may be placed in the bioreactor with Dulbecco's medium and supplemented with 5% human serum albumin either already in the bioreactor, added simultaneously to the bioreactor, or added after the cord blood to the bioreactor.

A preferred cord blood mixture of the present invention comprises the following: CD34+/CD38− stem cells isolated from the buffy coat of a cord blood sample collected from one infant at C-section; and Dulbecco's medium which, with the CD34+/CD38− cells, is about 200 ml total volume. Even more preferably, G-CSF (Granulocyte-Colony Stimulating Factor) is included in the cord blood mixture. Preferably, G-CSF is present in an amount sufficient to stimulate TVEMF-expansion of cord blood stem cells. Even more preferably, the amount of G-CSF present in the cord blood mixture prior to TVEMF-expansion is about 25 to about 200 ng/ml mixture, more preferably about 50 to about 150 ng/ml, and even more preferably about 100 ng/ml.

The TVEMF-bioreactor vessel (containing the cord blood mixture including the cord blood stem cells) is rotated at a speed that provides for suspension of the cord blood stem cells to maintain their three-dimensional geometry and their cell-to-cell support and cell-to-cell geometry. Preferably, the rotational speed is 5-120 rpm; more preferably, from 10-30 rpm. During the time that the cells are in the TVEMF-bioreactor, they are preferably fed nutrients and fresh media (DMEM and 5% human serum albumin), exposed to hormones, cytokines, and/or growth factors (preferably G-CSF); and toxic materials are removed. The toxic materials removed from cord blood cells in a TVEMF-bioreactor include the toxic granular material of dying cells and the toxic material of granulocytes and macrophages. The TVEMF-expansion of the cells is controlled so that the cells preferably expand (increase in number per volume, or concentration) at least seven times in a sufficient amount of time. Preferably, cord blood stem cells (with other cells, if present) undergo TVEMF-expansion for at least 4 days, preferably about 7 to about 14 days, more preferably about 7 to about 10 days, even more preferably about 7 days. TVEMF-expansion may continue in a TVEMF-bioreactor for up to 160 days. While TVEMF-expansion may occur for even longer than 160 days, such a lengthy expansion is not a preferred embodiment of the present invention.

Preferably, TVEMF-expansion is carried out in a TVEMF-bioreactor at a temperature of about 26 C to about 41 C, and more preferably, at a temperature of about 37 C.

One method of monitoring the overall expansion of cells undergoing TVEMF-expansion is by visual inspection. Cord blood stem cells are typically dark red in color. Preferably, the medium used to form the cord blood mixture is light or clear in color. Once the bioreactor begins to rotate and the TVEMF is applied, the cells preferably cluster in the center of the bioreactor vessel, with the medium surrounding the colored cluster of cells. Oxygenation and other nutrient additions often do not cloud the ability to visualize the cell cluster through a visualization (typically clear plastic) window built into the bioreactor. Formation of the cluster is important for helping the stem cells maintain their three-dimensional geometry and cell-to-cell support and cell-to-cell geometry; if the cluster appears to scatter and cells begin to contact the wall of the bioreactor vessel, the rotational speed is increased (manually or automatically) so that the centralized cluster of cells may form again. A measurement of the visualizable diameter of the cell cluster taken soon after formation may be compared with later cluster diameters, to indicate the approximate number increase in cells in the TVEMF-bioreactor. Measurement of the increase in the number of cells during TVEMF expansion may also be taken in a number of ways, as known in the art. An automatic sensor could also be included in the TVEMF-bioreactor to monitor and measure the increase in cluster size.

The TVEMF-expansion process may be carefully monitored, for instance by a laboratory expert, who will check cell cluster formation to ensure the cells remain clustered inside the bioreactor and will increase the rotation of the bioreactor when the cell cluster begins to scatter. An automatic system for monitoring the cell cluster and viscosity of the cord blood mixture inside the bioreactor may also monitor the cell clusters. A change in the viscosity of the cell cluster may become apparent about 2 days after beginning the TVEMF-expansion process, and the rotational speed of the TVEMF-bioreactor may be increased around that time. The TVEMF-bioreactor speed may vary throughout TVEMF-expansion. Preferably, the rotational speed is timely adjusted so that the cells undergoing TVEMF-expansion do not contact the sides of the TVEMF-bioreactor vessel.

Also, the laboratory expert may, for instance once a day, or once every two days, manually (for instance with a syringe) insert fresh media and preferably other desired additives such as nutrients and growth factors, as discussed above, into the bioreactor, and draw off the old media containing cell wastes and toxins. Also, fresh media and other additives may be automatically pumped into the TVEMF-bioreactor during TVEMF-expansion, and wastes automatically removed.

Cord blood stem cells may increase to at least seven times their original number about 7 to about 14 days after being placed in the TVEMF-bioreactor and TVEMF-expanded. Preferably, the TVEMF-expansion lasts about 7 to 10 days, and more preferably about 7 days. Measurement of the number of stem cells does not need to be taken during TVEMF-expansion therefore. As indicated above and throughout this application, TVEMF-expanded cord blood stem cells of the present invention have essentially the same three-dimensional geometry and cell-to-cell support and cell-to-cell geometry as naturally-occurring, non-TVEMF-expanded cord blood stem cells.

Another embodiment of the present invention relates to an ex vivo mammalian cord blood stem cell composition that functions to assist a body system or tissue to repair, replenish and regenerate tissue, for example, the tissues described throughout this application. The composition comprises TVEMF-expanded cord blood stem cells, preferably in an amount of at least seven times the number per volume of cord blood stem cells per volume as in the cord blood from which it originated. For instance, preferably, if a number X of cord blood stem cells was placed in a certain volume into a TVEMF-bioreactor, then after TVEMF-expansion, the number of cord blood stem cells from that same volume of cord blood stem cells place into the TVEMF-bioreactor will be at least 7×. While this at-least-seven-times-expansion is not necessary for this invention to work, this expansion is particularly preferred for therapeutic purposes. For instance, the TVEMF-expanded cells may be only in amount of 2 times the number of cord blood stem cells in the naturally-occuring cord blood, if desired. Preferably, TVEMF-expanded cells are in a range of about 4 times to about 25 times the number per volume of cord blood stem cells in naturally-occurring cord blood. The present invention is also directed to a composition comprising cord blood stem cells from a mammal, wherein said cord blood stem cells are present in a number per volume that is at least 7 times greater than naturally-occurring cord blood from the mammal; and wherein the cord blood stem cells have a three-dimensional geometry and cell-to-cell support and cell-to-cell geometry that is essentially the same as stem cells of the naturally-occurring cord blood. A composition of the present invention may include a pharmaceutically acceptable carrier; plasma, blood, albumin, cell culture medium, growth factor, copper chelating agent, hormone, buffer or cryopreservative. “Pharmaceutically acceptable carrier” means an agent that will allow the introduction of the stem cells into a mammal, preferably a human. Such carrier may include substances mentioned herein, including in particular any substances that may be used for blood transfusion, for instance blood, plasma, albumin, preferably from the mammal to which the composition will be introduced. The term “introduction” of a composition to a mammal is meant to refer to “administration” of a composition to an animal. “Acceptable carrier” generally refers to any substance the cord blood stem cells of the present invention may survive in, ie that is not toxic to the cells, whether after TVEMF-expansion, prior to or after cryopreservation, prior to introduction (administration) into a mammal. Such carriers are well known in the art, and may include a wide variety of substances, including substances described for such a purpose throughout this application. For instance, plasma, blood, albumin, cell culture medium, buffer and cryopreservative are all acceptable carriers of this invention. The desired carrier may depend in part on the desired use

Other expansion methods known in the art (none of which use TVEMF) do not provide an expansion of cord blood stem cells in this amount of at least 7 times that of naturally-occurring cord blood while still maintaining the cord blood stem cells three-dimensional geometry and cell-to-cell support. TVEMF-expanded cord blood stem cells have essentially the same, or maintain, the three-dimensional geometry and the cell-to-cell support and cell-to-cell geometry as the cord blood from which they originated. The composition comprises TVEMF-expanded cord blood stem cells, preferably in a suspension of Dulbecco's medium or in a solution ready for cryopreservation. The composition is preferably free of toxic granular material, for example, dying cells and the toxic material or content of granulocytes and macrophages. The composition may be a cryopreserved composition comprising TVEMF-expanded cord blood stem cells by decreasing the temperature of the composition to a temperature of from −120° C. to −196° C. and maintaining the cryopreserved composition at that temperature range until needed for therapeutic or other use. As discussed below, preferably, as much toxic material as is possible is removed from the composition prior to cryopreservation.

Another embodiment of the present invention relates to a method of regenerating tissue and/or treating diseases such as auto-immune diseases (as discussed above) with a composition of TVEMF-cord blood stem cells, either having undergone cryopreservation or soon after TVEMF-expansion is complete. The cells may be introduced into a mammalian body, preferably human, for instance injected intravenously or directly into the tissue to be repaired, allowing the body's natural system to repair and regenerate the tissue. Preferably, the composition introduced into the mammalian body is free of toxic material and other materials that may cause an adverse reaction to the administered TVEMF-expanded cord blood stem cells. The method (and composition) can potentially be used to repair a mammalian, preferably human, vital organ and other tissue, with such potential use including but not limited to liver tissue, heart tissue, hematopoietic tissue, blood vessels, skin tissue, muscle tissue, gut tissue, pancreatic tissue, central nervous system cells, bone, cartilage tissue, connective tissue, pulmonary tissue, spleen tissue, brain tissue and other body tissue. The cells are readily available for treatment or research where such treatment or research requires the individual's blood cells, especially if a disease has occurred and cells free of the disease are needed.

### Operative Method—Cryopreservation

As mentioned above, cord blood is collected for instance during the birth (even more preferably at a Caesarean section) of a baby mammal, preferably a human infant. Red blood cells are preferably removed from the cord blood. The cord blood stem cells (with other cells and media as desired) are placed in a TVEMF-bioreactor, subjected to a time varying electromagnetic force and expanded. After expansion, the cells may be cryogenically preserved. Further details relating to cryopreservation of a TVEMF-expanded cord blood stem cell composition are provided herein and in particular below.

After TVEMF-expansion, the TVEMF-expanded cells, including TVEMF-expanded cord blood stem cells, are preferably transferred into at least one cryopreservation container containing at least one cryoprotective agent. The TVEMF-expanded cord blood stem cells are preferably first washed with a solution (for instance, a buffer solution or the desired cryopreservative solution) to remove media and other components present during TVEMF-expansion, and then put in a solution that allows for cryopreservation of the cells. The cells are transferred to an appropriate cryogenic container and the container decreased in temperature to generally from −120° C. to −196° C., preferably about −130 to about −150 C, and maintained at that temperature. When needed, the temperature of the cells (ie the temperature of the cryogenic container) is raised to a temperature compatible with introduction into the human body (generally from around room temperature to around body temperature), and the TVEMF-expanded cells are introduced into a mammalian body, preferably human, for instance as discussed above.

Freezing cells is ordinarily destructive. On cooling, water within the cell freezes. Injury then occurs by osmotic effects on the cell membrane, cell dehydration, solute concentration, and ice crystal formation. As ice forms outside the cell, available water is removed from solution and withdrawn from the cell, causing osmotic dehydration and raised solute concentration that eventually destroys the cell. (For a discussion, see Mazur, P., 1977, Cryobiology 14:251-272.)

Different materials have different freezing points. Preferably, a cord blood stem cell composition ready for cryopreservation contains as few contaminating substances as possible, to minimize cell wall damage from the crystallizaton and freezing process.

These injurious effects can be circumvented by (a) use of a cryoprotective agent, (b) control of the freezing rate, and (c) storage at a temperature sufficiently low to minimize degradative reactions.

The inclusion of cryopreservation agents is preferred in the present invention. Cryoprotective agents which can be used include but are not limited to a sufficient amount of dimethyl sulfoxide (DMSO) (Lovelock, J. E. and Bishop, M. W. H., 1959, Nature 183:1394-1395; Ashwood-Smith, M. J., 1961, Nature 190:1204-1205), glycerol, polyvinylpyrrolidine (Rinfret, A. P., 1960, Ann. N.Y. Acad. Sci. 85:576), polyethylene glycol (Sloviter, H. A. and Ravdin, R. G., 1962, Nature 196:548), albumin, dextran, sucrose, ethylene glycol, i-erythritol, D-ribitol, D-mannitol (Rowe, A. W., et al., 1962, Fed. Proc. 21:157), D-sorbitol, i-inositol, D-lactose, choline chloride (Bender, M. A., et al., 1960, J. Appl. Physiol. 15:520), amino acid-glucose solutions or amino acids (Phan The Tran and Bender, M. A., 1960, Exp. Cell Res. 20:651), methanol, acetamide, glycerol monoacetate (Lovelock, J. E., 1954, Biochem. J. 56:265), and inorganic salts (Phan The Tran and Bender, M. A., 1960, Proc. Soc. Exp. Biol. Med. 104:388; Phan The Tran and Bender, M. A., 1961, in Radiobiology, Proceedings of the Third Australian Conference on Radiobiology, Ilbery, P. L. T., ed., Butterworth, London, p. 59). In a preferred embodiment, DMSO is used. DMSO, a liquid, is nontoxic to cells in low concentration. Being a small molecule, DMSO freely permeates the cell and protects intracellular organelles by combining with water to modify its freezability and prevent damage from ice formation. Adding plasma (ie, to a concentration of 20-25%) can augment the protective effect of DMSO. After addition of DMSO, cells should be kept at 0° C. until freezing, since DMSO concentrations of about 1% are toxic at temperatures above 4° C. My selected preferred cryoprotective agents are, in combination with TVEMF-expanded cord blood stem cells, 20 to 40% dimethyl sulfoxide solution in 60 to 80% amino acid-glucose solution, or 15 to 25% hydroxyethyl starch solution, or 4 to 6% glycerol, 3 to 5% glucose, 6 to 10% dextran T10, or 15 to 25% polyethylene glycol or 75 to 85% amino acid-glucose solution.

While other substances, other than cord blood cells and a cryoprotective agent, may be present in the present invention, preferably cryopreservation of a TVEMF-expanded cord blood stem cell composition of the present invention occurs with as few other substances as possible, for instance for reasons such as those discussed regarding the mechanism of freezing, above.

Preferably, a TVEMF-expanded cord blood stem cell composition of the present invention is cooled to a temperature in the range of about −120 C to about −196 C, preferably about −130 C to about −196 C, and even more preferably about −130 C to about −150 C.

A controlled slow cooling rate is critical. Different cryoprotective agents (Rapatz, G., et al., 1968, Cryobiology 5(1):18-25) and different cell types have different optimal cooling rates (see e.g. Rowe, A. W. and Rinfret, A. P., 1962, Blood 20:636; Rowe, A. W., 1966, Cryobiology 3(1):12-18; Lewis, J. P., et al., 1967, Transfusion 7(1):17-32; and Mazur, P., 1970, Science 168:939-949 for effects of cooling velocity on survival of peripheral cells (and on their transplantation potential)). The heat of fusion phase where water turns to ice should be minimal. The cooling procedure can be carried out by use of, e.g., a programmable freezing device or a methanol bath procedure.

Programmable freezing apparatuses allow determination of optimal cooling rates and facilitate standard reproducible cooling. Programmable controlled-rate freezers such as Cryomed or Planar permit tuning of the freezing regimen to the desired cooling rate curve. Other acceptable freezers may be, for example, Sanyo Modl MDF-1155ATN-152C and Model MDF-2136ATN-135C, Princeton CryoTech TEC 2000. For example, for peripheral blood cells or CD34+/CD38− cells in 10% DMSO and 20% plasma, the optimal rate is 1 to 3° C./minute from 0° C. to −200° C.

In a preferred embodiment, this cooling rate can be used for the cells of the invention. The cryogenic container holding the cells must be stable at cryogenic temperatures and allow for rapid heat transfer for effective control of both freezing and thawing. Sealed plastic vials (e.g., Nunc, Wheaton cryules) or glass ampules can be used for multiple small amounts (1-2 ml), while larger volumes (100-200 ml) can be frozen in polyolefin bags (e.g., Delmed) held between metal plates for better heat transfer during cooling. (Bags of bone marrow cells have been successfully frozen by placing them in −80° C. freezers that, fortuitously, gives a cooling rate of approximately 3° C./minute).

In an alternative embodiment, the methanol bath method of cooling can be used. The methanol bath method is well suited to routine cryopreservation of multiple small items on a large scale. The method does not require manual control of the freezing rate nor a recorder to monitor the rate. In a preferred aspect, DMSO-treated cells are precooled on ice and transferred to a tray containing chilled methanol that is placed, in turn, in a mechanical refrigerator (e.g., Harris or Revco) at −130° C. Thermocouple measurements of the methanol bath and the samples indicate the desired cooling rate of 1 to 3° C./minute. After at least two hours, the specimens have reached a temperature of −80° C. and can be placed directly into liquid nitrogen (−196° C.) for permanent storage.

After thorough freezing, TVEMF-expanded stem cells can be rapidly transferred to a long-term cryogenic storage vessel. In a preferred embodiment, samples can be cryogenically stored in liquid nitrogen (−196° C.) or its vapor (−165° C.). The storage temperature should be below −120° C., preferably below −130° C. Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum.

The preferred apparatus and procedure for the cryopreservation of the cells is that manufactured by Thermogenesis Corp., Rancho Cordovo, Calif., utilizing their procedure for lowering the cell temperature to below −130° C. The cells are held in a Thermogenesis plasma bag during freezing and storage.

After the temperature of the TVEMF-expanded cord blood stem cell composition is reduced to below −120° C., preferably below −130° C., they may be held in an apparatus such as a Thermogenesis freezer. Their temperature is maintained at a temperature of about −120° C. to −196° C., preferably −130° C. to −150° C. The temperature of a cryopreserved TVEMF-expanded cord blood stem cell composition of the present invention should not be about −120 C for a prolonged period of time.

A cryopreserved TVEMF-expanded cord blood stem cell composition according to the present invention may be frozen for an indefinite period of time, to be thawed when needed. For instance, a composition may be frozen for up to 18 years. Even longer time periods may work, perhaps even as long as the lifetime of an infant donor.

When needed, bags with the cells therein may be placed in a thawing system such as a Thermogenesis Plasma Thawer or other apparatus in the Thermoline Thawer series. The temperature of the cryopreserved composition is raised to room temperature. In another preferred method of thawing the cells mixed with a cryoprotective agent, bags having a cryopreserved TVEMF-expanded cord blood stem cell composition of the present invention, stored in liquid nitrogen, may be placed in the gas phase of liquid nitrogen for 15 minutes, exposed to ambient air room temperature for 5 minutes, and finally thawed in a 37° C. water bath as rapidly as possible. The thawed bags are immediately diluted with an equal volume of a solution containing 2.5% (weight/volume) human serum albumin and 5% (weight/volume) Dextran 40 (Solplex 40; Sifra, Verona, Italy) in isotonic salt solution and subsequently centrifuged at 400 g for ten minutes. The supernatant is removed and the sedimented cells are resuspended in fresh albumin/Dextran solution. See Rubinstein, P. et al., Processing and cryopreservation of placental/umbilical cord blood for unrelated bone marrow reconstitution. Proc. Natl. Acad. Sci. 92:10119-1012 (1995) for Removal of Hypertonic Cryoprotectant; a variation on this preferred method of thawing cells can be found in Lazzari, L. et al., Evaluation of the effect of cryopreservation on ex vivo expansion of hematopoietic progenitors from cord blood. Bone Marrow Trans. 28:693-698 (2001).

After the cells are raised in temperature to room temperature, they are available for research or regeneration therapy. The thawed TVEMF-expanded cord blood stem cell composition may be introduced directly into a mammal, preferably human, or used in its thawed form in desired research. Various additives may be added to the thawed compositions (or to a non-cryopreserved TVEMF-expanded cord blood stem cell composition) prior to introduction into a mammalian body, preferably soon to immediately prior to such introduction. Such additives include but are not limited to a growth factor, a copper chelating agent, a cytokine, a hormone, a suitable buffer or diluent. Preferably, G-CSF is added. Even more preferably, for humans, G-CSF is added in an amount of about 20 to about 40 micrograms/kg body weight, and even more preferably in an amount of about 30 micrograms/kg body weight. Also, prior to introduction, the TVEMF-expanded cord blood stem cell composition may be mixed with the mammal's own, or a suitable donor's, plasma, blood or albumin, or other materials that may accompany blood transfusions. The thawed cord blood stem cells can be used for instance to test to see if there is an adverse reaction to a pharmaceutical that is desired to be used for treatment or they can be used for treatment.

While the FDA has not approved use of expanded cord blood stem cells for regeneration of tissue in the United States, such approval appears to be imminent. Since the collection of cord blood can only be accomplished within a short time period of birth, if they are going to be collected for future use, they must be collected and expanded and stored for later research and possible later uses. Direct injection of a sufficient amount of expanded cord blood stem cells should be able to be used to regenerate vital organs such as the heart, liver, pancreas, skin, muscle, gut, spleen, brain, etc.

A TVEMF-expanded cord blood stem cell composition of the present invention should be introduced into a mammal, preferably a human, in an amount sufficient to achieve tissue repair or regeneration, or to treat a desired disease or condition. Preferably, at least 20 ml of a TVEMF-expanded cord blood stem cell composition having 10⁷ to 10⁹ stem cells per ml is used for any treatment, preferably all at once, in particular where a traumatic injury has occurred and immediate tissue repair needed. This amount is particularly preferred in a 75-80 kg human. The amount of TVEMF-expanded cord blood stem cells in a composition being introduced into the source mammal is inherently related to the number of cells present in the source cord blood material (for instance, the amount of stem cells present in one infant's cord blood). A preferred range of TVEMF-expanded cord blood stem cells introduced into a patient may be, for instance, about 10 ml to about 50 ml of a TVEMF-expanded cord blood stem cell composition having 10⁷ to 10⁹ stem cells per ml, or potentially even more. While it is understood that a high concentration of any substance, administered to a mammal, may be toxic or even lethal, it is unlikely that introducing all of a mammal's cord blood stem cells, for instance after TVEMF-expansion at least 7 times, will cause an overdose in TVEMF-expanded cord blood stem cells. Where cord blood from several donors is used, the number of cord blood stem cells introduced into a mammal may be higher. Also, the dosage of TVEMF-cells that may be introduced to the patient is not limited by the amount of cord blood provided from collection from one individual; multiple administrations, for instance once a day or twice a day, or once a week, or other administration time frames, may more easily be used. Also, where a tissue is to be treated, the type of tissue may warrant the use of as many TVEMF-expanded cord blood stem cells as are available. For instance, liver is easiest to treat.

It is to be understood that, while the embodiment described above generally relates to cryopreserving TVEMF-expanded cord blood stem cells, TVEMF-expansion may occur after thawing of already cryopreserved, non-expanded, or non-TVEMF-expanded, cord blood stem cells. Many cord blood banks, for instance, have cryopreserved compositions comprising cord blood stem cells in frozen storage, in case such is needed at some point in time. Such compositions may be thawed according to conventional methods and then TVEMF-expanded as described herein, including variations in the TVEMF-process as described herein. Thereafter, such TVEMF-expanded cord blood stem cells are considered to be compositions of the present invention, as described above. TVEMF-expansion prior to cryopreserving is preferred, for instance as if a traumatic injury occurs, a patient's cord blood stem cells have already been expanded and do not require precious extra days to prepare.

Also, while not preferred, it should be noted that TVEMF-expanded cord blood stem cells of the present invention may be cryopreserved, and then thawed, and then if not used, cryopreserved again.

Also, it is to be understood that the TVEMF-expanded cord blood stem cells of the present application may be introduced into a mammal, preferably the source mammal (mammal that is the source of the cord blood), after TVEMF-expansion, with or without cryopreservation.

Thawed TVEMF-expanded cord blood stem cells may be used for research for possible cures for the following diseases:

I. Diseases resulting from a failure or dysfunction of normal blood cell production and maturation, hyperproliferative stem cell disorders, aplastic anemia, pancytopenia, thrombocytopenia, red cell aplasia, Blackfan-Diamond syndrome due to drugs, radiation, or infection, idiopathic;

II. Hematopoietic malignancies, acute lymphoblastic (lymphocytic) leukemia, chronic lymphocytic leukemia, acute myelogenous leukemia, chronic myelogenous leukemia, acute malignant myelosclerosis, multiple myeloma, polycythemia vera, agnogenic myelometaplasia, Waldenstrom's macroglobulinemia, Hodgkin's lymphoma, non-Hodgkins's lymphoma;

III. Immunosuppression in patients with malignant, solid tumors, malignant melanoma, carcinoma of the stomach, ovarian carcinoma, breast carcinoma, small cell lung, carcinoma, retinoblastoma, testicular carcinoma, glioblastoma, rhabdomyosarcoma, neuroblastoma, Ewing's sarcoma, lymphoma;

IV. Autoimmune diseases, rheumatoid arthritis, diabetes type I, chronic hepatitis, multiple sclerosis, and systemic lupus erythematosus;

V. Genetic (congenital) disorders, anemias, familial aplastic, Fanconi's syndrome, Bloom's syndrome, pure red cell aplasia (PRCA), dyskeratosis congenital, Blackfan-Diamond syndrome, congenital dyserythropoietic syndromes I-IV, Chwachmann-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phosphate dehydrogenase), variants 1,2,3, pyruvate kinase deficiency, congenital erythropoietin sensitivity, deficiency, sickle cell disease and trait, thalassemia alpha, beta, gamma met-hemoglobinemia, congenital disorders of immunity, severe combined immunodeficiency disease, (SCID), bare lymphocyte syndrome, ionophore-responsive combined, immunodeficiency, combined immunodeficiency with a capping abnormality, nucleoside phosphorylase deficiency, granulocyte actin deficiency, infantile agranulocytosis, Gaucher's disease, adenosine deaminase deficiency, Kostmann's syndrome, reticular dysgenesis, congenital leukocyte dysfunction syndromes; and

VI. Others including osteopetrosis, myelosclerosis, acquired hemolytic anemias, acquired immunodeficiencies, infectious disorders causing primary or secondary immunodeficiencies, bacterial infections (e.g., Brucellosis, Listerosis, tuberculosis, leprosy), parasitic infections (e.g., malaria, Leishmaniasis), fungal infections, disorders involving disproportions in lymphoid cell sets and impaired immune functions due to aging phagocyte disorders, Kostmann's agranulocytosis, chronic granulomatous disease, Chediak-Higachi syndrome, neutrophil actin deficiency, neutrophil membrane GP-180 deficiency, metabolic storage diseases, mucopolysaccharidoses, mucolipidoses, miscellaneous disorders involving immune mechanisms, Wiskott-Aldrich Syndrome, alpha 1-antitrypsin deficiency.

During the entire process of expansion, preservation, and thawing, cord blood stem cells of the present invention maintain their three-dimensional geometry and their cell-to-cell support and cell-to-cell geometry.

While preferred embodiments have been herein described, those skilled in the art will understand the present invention to include various changes and modifications. The scope of the invention is not intended to be limited to the above-described embodiments.

I claim:

1. Cord blood stem cells from a mammal,
wherein said cord blood stem cells are in a number per volume that is at least 7 times greater than naturally-occurring cord blood; and
wherein the cord blood stem cells have a three-dimensional geometry and cell-to-cell support and cell-to-cell geometry that is essentially the same as stem cells of naturally-occurring cord blood.

2. A composition comprising the cord blood stem cells of claim 1 and an acceptable carrier.

3. A composition of claim 2, wherein said acceptable carrier is at least one of the group consisting of plasma, blood, albumin, cell culture medium, growth factor, copper chelating agent, hormone, buffer and cryopreservative.

4. The composition of claim 3, wherein said growth factor is G-CSF.

5. The composition of claim 2, wherein said composition is at a temperature sufficient to cryogenically preserve the cord blood stem cells.

6. The composition according to claim 2, wherein a cryopreservative is present in an amount sufficient for cryopreservation of said cells, and wherein said composition is at a temperature of from about −120° C. to about −196° C.

7. The composition according to claim 6, wherein said temperature is from about −130° C. to about −150° C.

8. The composition according to claim 6, further comprising a pharmaceutically acceptable carrier.

9. The composition according to claim 3, wherein said composition comprises a cryopreservative selected from the group consisting of 20 to 40% dimethyl sulfoxide solution in 60 to 80% amino acid-glucose solution; 15 to 25% hydroxyethyl starch solution; 4 to 6% glycerol, 3 to 5% glucose and 6 to 10% dextran T10; 15 to 25% polyethylene glycol; and 75 to 85% amino acid-glucose solution.

10. The composition of claim 2, wherein said composition is free of toxic material.

11. Cord blood stem cells from a mammal, wherein said cord blood stem cells are TVEMF-expanded.

12. The TVEMF-expanded cord blood stem cells of claim 11, wherein the number of TVEMF-expanded cord blood stem cells per volume is at least 2 times greater than the number of stem cells per volume of the cord blood from which the TVEMF-expanded cord blood stem cells are derived.

13. The TVEMF-expanded cord blood stem cells of claim 12, wherein the number of TVEMF-expanded cord blood stem cells per volume is at least 7 times greater.

14. A composition comprising the cord blood stem cells of claim 13 and an acceptable carrier.

15. A composition comprising the TVEMF-expanded cord blood stem cells of claim 13 wherein said composition further comprises at least one of the group consisting of plasma, blood, albumin, cell culture medium, growth factor, copper chelating agent, hormone, buffer and cryopreservative.

16. The composition of claim 15, wherein said growth factor is G-CSF.

17. The composition according to claim 14, wherein said composition further comprises cryopreservative selected from the group consisting of 20 to 40% dimethyl sulfoxide solution in 60 to 80% amino acid-glucose solution; 15 to 25% hydroxyethyl starch solution; 4 to 6% glycerol, 3 to 5% glucose and 6 to 10% dextran T10; 15 to 25% polyethylene glycol; and 75 to 85% amino acid-glucose solution.

18. The composition of claim 14, wherein said composition is at a temperature sufficient to cryogenically preserve the cord blood stem cells.

19. The composition according to claim 14, wherein a cryopreservative is present and wherein said composition is at a temperature of from about −120° C. to about −196° C.

20. The composition according to claim 14, wherein said temperature is from about −130° C. to about −150° C.

21. The composition of claim 14, wherein said composition is free of toxic material.

22. A process for preparing a cord blood stem cell composition comprising the steps of:
a. placing a cord blood mixture in a culture chamber of a TVEMF-bioreactor; and
b. subjecting the cord blood mixture to a TVEMF and TVEMF-expanding the cord blood stem cells to prepare the cord blood stem cell composition.

23. The process according to claim 22, wherein said TVEMF is about 0.05 to about 6.0 gauss.

24. The process according to claim 22, wherein said TVEMF-expanding continues until the number per volume of TVEMF-expanded cord blood stem cells is more than 7 times the number per volume of cord blood stem cells placed in the TVEMF-bioreactor.

25. The process according to claim 22, further comprising collecting cord blood from a mammal prior to placing the cord blood mixture in a TVEMF-bioreactor.

26. The process of claim 25, wherein said mammal is human.

27. The process according to claim 22, further comprising collecting thawed cryopreserved cord blood from a cord blood storage facility prior to adding the cord blood to the cord blood mixture.

28. The process of claim 22, further comprising a step of removing toxic material from the cord blood mixture prior to TVEMF-expansion.

29. The process of claim 22, wherein the TVEMF-bioreactor has an integral TVEMF source.

30. The process of claim 22, wherein the TVEMF-bioreactor has an adjacent TVEMF source.

31. The process of claim 22, wherein the cord blood mixture comprises CD34+/CD38− cord blood stem cells separated from other cord blood components.

32. The process of claim 22, wherein the cord blood mixture comprises a buffy coat separated from other cord blood components.

33. The process of claim 22, wherein the cord blood mixture comprises cord blood free of red blood cells.

34. The process of claim 22, further comprising the steps of transferring the TVEMF-expanded cells of the cord blood stem cell composition into a cryogenic container having a temperature, and lowering the temperature of the cryogenic container to a temperature of from −120° C. to −196° C. at a controlled rate.

35. The process of claim 34, further comprising a step of removing toxic material from the cord blood stem cell composition prior to lowering the temperature to a temperature of from −120° C. to −196° C. at a controlled rate.

36. The process of claim 34, further comprising, after the step of lowering the temperature, a step of maintaining the temperature of the cryogenic container to a temperature of from −120° C. to −196° C., for a period of time.

37. The process of claim 36, wherein said period of time is at least 1 year.

38. The process of claim 36, further comprising, after said lowering and maintaining of temperature, a step of increasing the temperature of the cryogenic container at a controlled rate to a temperature suitable for introducing the cord blood stem cell composition to a mammal.

39. The process of claim 38, wherein toxic material has been removed from said increased temperature cord blood stem cell composition.

40. The process of claim 34, further comprising the step of adding a cryopreservative to the TVEMF-expanded cells of the cord blood stem cell composition.

41. A composition comprising cord blood stem cells and an acceptable carrier prepared by the process according to claim 22.

42. A method of repairing tissue of a mammal comprising the step of administering to the mammal a therapeutically effective amount of a composition comprising the cord blood stem cells of claim 1 and a pharmaceutically acceptable carrier.

43. A method of repairing tissue of a mammal comprising the step of administering to the mammal a therapeutically effective amount of a composition comprising the TVEMF-expanded cord blood stem cells of claim 11 and a pharmaceutically acceptable carrier.

44. The method of claim 43, wherein the tissue to be repaired is human tissue.

45. The method of claim 44, wherein the tissue to be repaired is a vital organ.

46. The method of claim 44, wherein the tissue to be repaired is at least one selected from the group consisting of a liver tissue, heart tissue, hematopoietic tissue, blood vessels, skin tissue, muscle tissue, gut tissue, pancreatic tissue, central nervous system cells, bone, cartilage tissue, connective tissue, pulmonary tissue, spleen tissue and brain tissue.

47. The method of claim 43, wherein the amount of TVEMF-expanded cord blood stem cells to be administered to the mammal is at least 20 ml of a composition having 10⁷ to 10⁹ stem cells/ml.

48. A method of treating a disease of a mammal comprising the step of administering to the mammal a therapeutically effective amount of a composition comprising the cord blood stem cells of claim 1 and a pharmaceutically acceptable carrier.

49. A method of treating a disease of a mammal comprising the step of administering to the mammal a therapeutically effective amount of a composition comprising the TVEMF-expanded cord blood stem cells of claim 11 and a pharmaceutically acceptable carrier.

50. The method of claim 49, wherein the tissue to be repaired is human tissue.

51. The method of claim 50, wherein the tissue to be repaired is a vital organ.

52. The method of claim 50, wherein the tissue to be repaired is at least one selected from the group consisting of a liver tissue, heart tissue, hematopoietic tissue, blood vessels, skin tissue, muscle tissue, gut tissue, pancreatic tissue, central nervous system cells, bone, cartilage tissue, connective tissue, pulmonary tissue, spleen tissue and brain tissue.

53. The method of claim 49, wherein the amount of TVEMF-expanded cord blood stem cells to be administered to the mammal is at least 20 ml of a composition having 10⁷ to 10⁹ stem cells/ml.
